(19) United States
(12) Patent Application Publication
(10) Pub. No.: US 2006/0165790 A1
(43) Pub. Date: Jul. 27, 2006

(54) Multiparticulates

(76) Inventors: Malcolm Walden, Hardwick (GB); Geoffrey Gerard Hayes, Saffron Walden (GB); Hassan Mohammad, Littleport (GB); Harjit Tamber, Hitchin (GB); Steve Whitelock, Milton (GB); Vincenzo Martinelli, Cambridge (GB)

Correspondence Address: JONES DAY, 222 EAST 41ST ST, NEW YORK, NY, 10017, US

(21) Appl. No.: 11/314,464

(22) Filed: Dec. 20, 2005

Related U.S. Application Data
(63) Continuation-in-part of application No. PCT/GB04/02705, filed on Jun. 23, 2004.

(30) Foreign Application Priority Data
Dec. 20, 2004 (GB) GB 0427745.5
Jun. 27, 2003 (GB) GB 0315137.0
Feb. 12, 2004 (GB) GB 0403102.7
Jun. 16, 2004 (GB) GB 0413454.0

(51) Int. Cl. A61K 31/485; A61K 9/22

(52) U.S. Cl. 424/468; 514/282

(57) ABSTRACT

Multipartulates of oxycodone can be made by extrusion of a blend which suitably contains (a) oxycodone, (b) water-insoluble ammonium methacrylate copolymer, (c) plasticiser, (d) lubricant and (e) water permeability modifier.

The present invention relates to multiparticulates, and in particular to extruded multiparticulates which provide controlled release of oxycodone.

### BACKGROUND OF THE INVENTION

Oxycodone is 4,5-epoxy-14-hydroxy-3-methoxy-17-methylmorphinan-6-one and is derived from the opium alkaloid thebaine. It is a pure agonist opioid whose principal action is analgesia, and is usually administered as oxycodone hydrochloride. The hydrochloride salt of oxycodone is a white, odourless crystalline powder which dissolves freely in water (1 g in 6 to 7 ml).

Oxycodone is indicated for the treatment of moderate to severe pain. Controlled release oxycodone products enable management of pain when a continuous and around-the-clock supply of analgesic is needed for an extended period of time.

Formulations of oxycodone which provide controlled release of oxycodone are described for instance in WO 9310765. A granulation procedure is typically employed. In Example 3, a tablet containing 10 mg of oxycodone hydrochloride is prepared from a mix of oxycodone hydrochloride, lactose, povidone, Eudragit RS 30 D, triacetin, stearyl alcohol, talc and magnesium stearate. The same ingredients in adjusted amounts are employed in Example 4 to prepare tablets containing 20 mg oxycodone hydrochloride. The resultant products exhibit differing pharmacokinetic and pharmacodynamic properties.

Illustratively, the in vitro release rates of the 10 mg and 20 mg oxycodone tablets are given in WO 9310765 as follows:

% oxycodone released

| hour | 10 mg | 20 mg |
|---|---|---|
| 1 | 38.0 | 31 |
| 2 | 47.5 | 44 |
| 4 | 62.0 | 57 |
| 8 | 79.8 | 71 |
| 12 | 91.1 | 79 |
| 18 | 94.9 | 86 |
| 24 | 98.7 | 89 |

Tablets of this kind and with such release rates form the basis for a commercial product. Controlled release oxycodone tablets are available as OxyContin (Registered Trade Mark) Tablets, which are designed to provide controlled delivery of oxycodone over 12 hours.

Oxycodone is well absorbed from OxyContin® Tablets with an oral bioavailability of 60% to 87%. The relative oral bioavailability of OxyContin® Tablets to immediate-release oral dosage forms is 100%. Upon repeated dosing in normal volunteers in pharmacokinetic studies, steady-state levels were achieved within 24-36 hours.

Dose proportionality has been established for 10 mg, 20 mg, 40 mg, 80 mg, and 160 mg tablet strengths with respect to both peak plasma levels (Cₘₐₓ) and extent of absorption (bioavailability), AUC, as indicated by the following data:

Mean [% coefficient variation]

| | | | | | Trough |
|---|---|---|---|---|---|
| | Dosage | AUC | Cmax | Tmax | Conc. |
| Regimen | Form | (ng · hr/mL)* | (ng/mL) | hrs) | (ng/mL) |
| Single | 10 mg | 100.7 | 10.6 | 2.7 | n.a. |
| Dose | OxyContin ® | [26.6] | [20.1] | [44.1] | |
| | Tablets | | | | |
| | 20 mg | 207.5 | 21.4 | 3.2 | n.a. |
| | OxyContin ® | [35.9] | [36.6] | [57.9] | |
| | Tablets | | | | |
| | 40 mg | 423.1 | 39.3 | 3.1 | n.a. |
| | OxyContin ® | [33.3] | [34.0] | [77.4] | |
| | Tablets | | | | |
| | 80 mg | 1085.5 | 98.5 | 2.1 | n.a. |
| | OxyContin ® | [32.3] | [32.1] | [52.3] | |
| | Tablets** | | | | |
| Multiple | 10 mg | 103.6 | 15.1 | 3.2 | 7.2 |
| Dose | OxyContin ® | [38.6] | [31.0] | [69.5] | [48.1] |
| | Tablets ql2h | | | | |
| | 5 mg | 99.0 | 15.5 | 1.6 | 7.4 |
| | immediate- | [36.2] | [28.8] | [49.7] | [50.9] |
| | release q6h | | | | |

*for single-dose AUC = AUC_{0-inf}, for multiple dose AUC = AUC_{0-T}
**data obtained while volunteers received naltrexone which can enhance absorption

Oxycodone is extensively metabolized and eliminated primarily in the urine as both conjugated and unconjugated metabolites. The apparent elimination half-life of oxycodone following the administration of OxyContin® Tablets was 4.5 hours compared to 3.2 hours for immediate-release oxycodone.

About 60% to 87% of an oral dose of oxycodone reaches the central compartment in comparison to a parenteral dose. This high oral bioavailability is due to low pre-systemic and/or first-pass metabolism. In normal volunteers, the t_{1/2} of absorption is 0.4 hours for immediate-release oral oxycodone. In contrast, OxyContin® Tablets exhibit a biphasic absorption pattern with two apparent absorption half-lives of 0.6 and 6.9 hours, which describes the initial release of oxycodone from the tablet followed by a prolonged release.

Alternative techniques exist for the manufacture of oxycodone formulations, apart from the granulation employed in the Examples of WO 9310765. Thus, multiparticulates of uniform dimensions with modified drug release properties can be manufactured by a technique referred to as melt extrusion technology. Melt extrusion is a solvent-free single-step process for manufacturing multiparticulates by extruding a softened blend, and is particularly useful for drug release modification. By selection of suitable polymers and additives, melt extrusion technology can be used both to enhance the solubility, and subsequently the bioavailability, of poorly water soluble drugs as well as to retard drug release of moderate to highly water soluble drugs for controlled release products.

The backbone of melt extrusion technology is the application of thermoplastic materials which act as binders for embedded drugs in solution or dispersion form within the matrix. Thermoplastic polymers with low glass transition temperatures (Tg) are preferred for processing by melt extrusion. Lower processing temperatures are also preferred with respect to the stability of heat sensitive drugs and other necessary excipients. Polymer glass transition temperatures can also be further reduced to facilitate processing at lower temperatures with optional addition of plasticisers.

Illustratively, WO 9614058 provides a sustained-release pharmaceutical formulation, comprising a melt-extruded blend of a therapeutically active agent, one or more materials selected from the group consisting of alkylcelluloses, acrylic and methacrylic acid polymers and copolymers, shellac, zein, hydrogenated castor oil, hydrogenated vegetable oil, and mixtures thereof; and one or more hydrophobic fusible carriers which provide a further retardant effect and are selected from the group consisting of natural or synthetic waxes, fatty acids, fatty alcohols, and mixtures thereof, the fusible carrier having a melting point from 30 to 200° C. The melt-extruded blend is divided into a unit dose containing an effective amount of said therapeutically active agent to render a desired therapeutic effect and providing a sustained-release of said therapeutically active agent for a time period of from about 8 to about 24 hours.

Furthermore, WO 9614058 describes a method of preparing a sustained-release pharmaceutical extrudate suitable for oral administration. The method comprises:

blending a therapeutically active agent together with (1) a material selected from the group consisting of alkylcelluloses, acrylic and methacrylic acid polymers and copolymers, shellac, zein, hydrogenated castor oil, hydrogenated vegetable oil, and mixtures thereof and (2) a fusible carrier selected from the group consisting of natural or synthetic waxes, fatty acids, fatty alcohols, and mixtures thereof; said retardant material having a melting point between 30-200° C. and being included in an amount sufficient to further slow the release of the therapeutically active agent;

heating said blend to a temperature sufficient to soften the mixture sufficiently to extrude the same;

extruding said heated mixture as a strand having a diameter of from 0.1-3 mm; cooling said strand; and dividing said strand to form non-spheroidal multi-particulates of said extrudate having a length from 0.1-5 mm; and

dividing said non-spheroidal multi-particulates into unit doses containing an effective amount of said therapeutically active agent, said unit dose providing a sustained-release of said therapeutically active agent for a time period of from about 8 to about 24 hours.

This method can be applied to oxycodone, an opioid analgesic, and typically employs a Eudragit polymethacrylate as the main retarding polymer in the matrix. The Eudragit polymethacrylates are widely employed in pharmaceutical compositions, notably to control release of an active ingredient. Thus, in some of the examples of WO 9614058, controlled release capsules or tablets with 20 mg of oxycodone hydrochloride are prepared by extrusion of a blend. In Examples 11 and 13, the oxycodone hydrochloride is blended with Eudragit RS PO, Eudragit L 100 and stearic acid. The blend in Example 12 additionally contains talc.

A need remains to provide a method of preparing multiparticulates of oxycodone which can be used to fill a capsule which can approximate to some or all of the pharmacokinetic and pharmacodynamic characteristics of OxyContin® Tablets. A related object of this invention is the provision of a process for preparing an oxycodone pharmaceutical composition which provides an oxycodone in vitro release profile that approximates to that of Examples 3 and 4 of WO 9310765.

### SUMMARY OF THE INVENTION

According to the present invention, we provide a plurality of particles of oxycodone, referred to as oxycodone multiparticulates.

In one aspect, we provide oxycodone multiparticulates with a high initial release of oxycodone, and a high total release of oxycodone. The release properties can be expressed in terms of release of oxycodone under controlled in vitro conditions which for example simulate human gastric fluids or the human intestinal environment. Release at a physiological pH, for example a pH of about 1.2 or about 6.8, can be tested. Test procedures can also be designed to reflect a switch from the stomach to the intestine during passage through the body.

In particular, we have found that the inclusion of a water permeability modifier can permit extrusion of multiparticulates of oxycodone which show some bioequivalence to OxyContin® Tablets. The multiparticulates can have pharmacokinetic and/or pharmacodynamic properties approximating to those of OxyContin® Tablets. In particular, the multiparticulates can have in vitro release rates that approximate to those of OxyContin® Tablets.

In a related aspect, we provide oxycodone multiparticulates comprising oxycodone usually in the form of a pharmaceutically acceptable salt, an ammonium methacrylate copolymer, a plasticiser, a lubricant and a water permeability modifier. Typically the water permeability modifier serves to modify the water permeability and enhance the drug release, especially in the later stages of the dissolution. The water permeability modifier can also serve to modulate the rate of secretion of the drug.

The oxycodone can be in the form of a pharmaceutically acceptable salt, preferably the hydrochloride, or the free base.

The multiparticulates are preferably obtainable by extrusion of an extrudable blend. Such an extrusion can be of the kind disclosed in WO 9614058 and referred to as a melt extrusion. In practice, the polymer softens but in practice might not melt.

The multiparticulates of this invention can be used as a fill in a capsule. Thus, the present invention provides a capsule suited for once or twice a day dosing. Other dosage forms of the controlled release formulation can be provided. The dosage form is preferably a unit dosage form, and preferably shows some bioequivalence to OxyContin® Tablets. The dosage form can have pharmacokinetic and/or pharmacodynamic properties approximating to those of OxyContin® Tablets. In particular, the dosage form can have in vitro release rates that approximate to those of OxyContin® Tablets.

In a further aspect of the invention, there is provided a method of treating a patient with a controlled release formulation of this invention. The method includes administering a dosage form of this invention to a patient in need of oxycodone analgesic therapy.

In a related aspect, we provide a process for preparing oxycodone multiparticulates which comprises extrusion of an extrudable blend of oxycodone usually in the form of a pharmaceutically acceptable salt. The blend includes a water permeability modifier to modify the water permeability, and suitably comprises an ammonium methacrylate copolymer, a plasticiser, a lubricant and the water permeability modifier.

### DETAILS OF THE INVENTION

The oxycodone multiparticulates of this invention preferably give in vitro release rates that approximate to those of OxyContin® Tablets. The release rates of OxyContin® Tablets are notable for a high initial release, and a high total release. Preferably the release of oxycodone is substantially independent of pH in the pH range of around 1 to around 7. To this end, substantially pH-independent release can mean that for a given formulation when tested in simulated intestinal fluid at pH 6.8, at any given time point the amount of oxycodone released as a percentage of the original amount of oxycodone in the formulation is substantially equal to the percentage amount of oxycodone released based on the original amount of oxycodone in the formulation when tested in simulated gastric fluid at pH 1.2. The release is substantially equal when the respective amounts differ by ±30%, more preferably ±20% and most preferably ±15%.

Unless otherwise indicated, we measure release rates by a specified method which involves using Ph.Eur. basket dissolution apparatus at 37° C., 100 rpm in 900 ml of USP simulated gastric fluid at pH 1.2 without enzyme. In one variation, the dissolution medium is simulated intestinal fluid at pH 6.8 without enzyme.

For simulated gastric fluid at pH 1.2, the oxycodone multiparticulates of this invention typically release at least 15% oxycodone after 1 hour, reflecting a high initial release. Preferably they release at least 20%, more preferably at least 25% and most preferably at least 35% of the oxycodone after 1 hour.

The oxycodone multiparticulates of this invention typically release at least 30% oxycodone after 2 hours, reflecting a high initial release. Preferably they release at least 40%, more preferably at least 50% and most preferably at least 55% of the oxycodone after 2 hours.

The oxycodone multiparticulates of this invention typically release at least 60% oxycodone after 4 hours, reflecting a high initial release. Preferably they release at least 70%, more preferably at least 75% and most preferably at least 80% of the oxycodone after 4 hours.

The oxycodone multiparticulates of this invention typically release at least 75% oxycodone after 10 hours, reflecting a high total release. Preferably they release at least 80%, more preferably at least 90% and most preferably at least 95% of the oxycodone after 10 hours.

Furthermore, at least 85% release of oxycodone after 8 hours is preferred. The oxycodone multiparticulates of this invention can release 100% oxycodone after 12 hours, reflecting a high total release.

The preferred multiparticulates of this invention contain (a) oxycodone, (b) water-insoluble ammonium methacrylate copolymer, (c) plasticiser, (d) lubricant and (e) water permeability modifier. With this selection of ingredients it becomes possible to prepare multiparticulates and thus capsules containing oxycodone and which mimic the in vitro and preferably the in vivo release characteristics of OxyContin® Tablets. In particular, the combination including a water permeability modifier enables an adequate initial release of oxycodone (early hours) whilst maintaining a high total release of the active ingredient in the later hours of dissolution.

Oxycodone hydrochloride is the preferred form of oxycodone, though other pharmaceutically acceptable salts can be used.

The water-insoluble ammonium methacrylate copolymer, also referred to as a water-insoluble ammonio methacrylate copolymer, is suitably Eudragit RS PO. It offers the following properties:
insoluble to poorly water soluble,
low aqueous porosity or permeability,
compatible with the drug and other additives,
extrudable at moderate temperatures or at lower temperatures in the presence of a suitable plasticiser,
stable for the intended storage time and conditions,
thermal stability.

In particular, Eudragit RS PO is a thermoplastic polymer of low water permeability which can significantly retard release of embedded oxycodone in its matrix. It is described as a pH independent polymer powder with low permeability for matrix formulations. It is a copolymer of acrylic and methacyrylic acid esters, with a low content of quaternary ammonium groups to control permeability, and an average molecular weight of around 150,000.

The plasticiser serves to soften the insoluble ammonium methacrylate copolymer to make it more easy to extrude the polymer. To this end, the typical plasticiser is miscible with the insoluble ammonium methacrylate copolymer to produce a decreased tensile strength, a lower softening temperature, and a decrease in the glass transition temperature, Tg, of the polymer. It serves to reduce cohesion by providing internal lubrication of the polymer. The plasticiser is normally chosen from water insoluble solids such as cetyl alcohol, stearyl alcohol and cetostearyl alcohol; water soluble solids such as sorbitol and sucrose and high molecular weight polyethylene glycol; water insoluble liquids such as dibutyl sebacate and tributyl citrate and water soluble liquids such as triethyl citrate, propylene glycol and low molecular weight polyethylene glycol. Stearyl alcohol is a preferred plasticiser. Another preferred plasticiser is a high molecular weight polyethylene glycol, preferably with a molecular weight in the range 4000 to 10000, such as PEG 6000.

The lubricant is a processing aid which reduces friction between the plasticised polymer blend and the internal surfaces of the extruder. It is normally a solid, and is suitably chosen from stearic acid, glyceryl behenate (predominantly glyceryl dibehenate), magnesium stearate, calcium stearate, talc and silicone dioxide (fused silica). The presence of lubricant in the extrusion formulation improves blending, kneading and conveying, and reduces adhesion forces. Smooth lubricated extrusion at low to moderate temperatures improves batch to batch reproducibility and reduces the strain on both the product and equipment. Stearic acid, possibly in the form of a salt, is a preferred lubricant. Another preferred lubricant is glyceryl behenate, which gives less pH sensitivity for in vitro release of oxycodone.

Plasticisers can often act as a lubricant, and lubricants can often act as a plasticiser.

The choice of plasticiser and lubricant will usually have an effect on the characteristics of the resultant extruded multiparticulates. For example, where the plasticiser is stearyl alcohol and the lubricant is stearic acid, the quantities and ratios with respect to each other and relative to the ammonium methacrylate copolymer can significantly modify the release rate of the drug. We have found that higher levels of stearyl alcohol reduce the Tg of the polymer blend and believe this reduction affects the rate of drug release. However, higher levels of stearic acid can also improve the mixing, kneading and extrusion as well as alter the release rate of oxycodone. We have found that higher ratios of stearic acid at only the expense of stearyl alcohol show a significant reduction of the rate and total oxycodone release.

The water permeability modifier modulates secretion of the drug from the dosage form. Typically the water permeability modifier serves to enhance the drug release, especially in the later stages of the dissolution, though we also envisage that the water permeability modifier might in some instances play a role in slowing release. Examples of agents used to modify the water permeability of the extruded multiparticulates include an insoluble hydrophilic wicking agent, a gelling agent which hydrates to form a gel to control the water movement, a high molecular weight polyethylene glycol such as PEG 6000, or a water permeable ammonium methacrylate copolymer such as Eudragit RL PO, also referred to as an ammonio methacrylate copolymer. Eudragit RL PO is described as a highly permeable pH independent polymer powder for matrix formulations. It is a copolymer of acrylic and methacyrylic acid esters, with a content of quaternary ammonium groups to provide permeability, and an average molecular weight of around 150,000.

For example, microcrystalline cellulose, high molecular weight hydrogels such as high viscosity hydroxypropylmethyl cellulose and high viscosity poly(ethylene oxide), and water permeable ammonium methacrylate copolymers may be used to enhance the total release of the active. In this last respect, the ammonium methacrylate copolymer employed as agent (e) to modify the water permeability is not the same polymer as the water insoluble ammonium methacrylate copolymer used as ingredient (b), being more water permeable due to different degrees of substitution by quaternary ammonium groups.

Microcrystalline cellulose improves water diffusion and exchange and thus enhances drug release. The microcrystalline cellulose acts as an insoluble but hydrophilic wicking agent. Alternatives to microcrystalline cellulose are croscarmellose sodium, crospovidone or sodium starch glycollate.

High molecular weight grade (high viscosity) hydroxypropylmethyl cellulose (HPMC) initially hydrates to form a thick gel to control the water movement. The hydrated gel then gradually dissolves and/or erodes over time leaving a porous and highly permeable structure. According to this hypothesis, it is believed that high viscosity HPMC does not significantly increase drug release at the earlier hours but enhances the release at later time points. Other gelling agents are candidates, including polyethylene oxide, pectin, locust bean gum or xanthan gum.

Eudragit RL PO is a highly water permeable analogue and can significantly enhance the release rate and total drug release.

Suitable percentage amounts for the ingredients (a) to (e) are given in the following table, based on the total weight of the five ingredients:

| | | | more |
|---|---|---|---|
| | typical | preferred | preferred |
| | range % | range % | range % |
| oxycodone as hydrochloride | 3 to 50 | 5 to 40 | 7.5 to 35 |
| insoluble ammonium | 25 to 85 | 35 to 75 | 50 to 65 |
| methacrylate copolymer | | | |
| plasticiser | 1 to 30 | 3 to 25 | 5 to 15 |
| lubricant | 1 to 25 | 2 to 25 | 2 to 25 |
| water permeability modifier | 1 to 40 | 1 to 30 | 1 to 20 |

As part of our investigations, we have identified the need to reduce the processing temperatures by optimising the component plasticiser/lubricant excipients. Furthermore, requirements for providing a twice-a-day capsule in 40 mg and 80 mg strengths using size 1 capsules led to further re-assessment of the drug load.

As a result, we now also identify the following suitable percentage amounts for the ingredients (a) to (e) given in the following table, based on the total weight of the five ingredients:

| | | | more |
|---|---|---|---|
| | typical | preferred | preferred |
| | range % | range % | range % |
| oxycodone as hydrochloride | 25 to 32 | 29 to 31 | about 30, |
| | | | for example 30.3 |
| insoluble ammonium | 25 to 85 | 35 to 75 | 45 to 70 |
| methacrylate copolymer | | | |
| plasticiser | 1 to 30 | 3 to 25 | 5 to 20 |
| lubricant | 1 to 25 | 2 to 25 | 2 to 10 |
| water permeability modifier | 1 to 40 | 1 to 30 | 1 to 15 |

Other additives may also be employed to produce multiparticulates within a set of predetermined specifications. Bulking agents, for example lactose, microcrystalline cellulose and calcium phosphate, are widely used pharmaceutical excipients and can be used in the present invention to modify the release rates and/or total release. Other release modifying agents may also be considered to modulate the release rate and/or enhance total release.

The preferred formulation contains oxycodone, preferably as the hydrochloride salt, Eudragit RS PO as water-insoluble ammonium methacrylate copolymer, stearyl alcohol as plasticiser, glyceryl behenate as lubricant, and Eudragit RL PO as water permeability modifier.

For manufacture of the multiparticulates of this invention, the ingredients are blended, and extruded. Details of such procedures are given in WO 9614058, which is incorporated herein in full by specific reference.

For the present invention, we prefer to employ a twin screw extruder, which can have co-rotating or counter-rotating screws. Essentially, the blend as a powder is fed by a feeder into the first segment of the barrel usually at relatively low temperature, for example 10-20° C., to ensure a constant powder flow to the high temperature barrels. The feeder provides a uniform current of the blend to the extruder. Consistency is desirable as irregular and variable feeding rates can produce multiparticulates with varying physical properties, such as density and porosity.

The preferred extruder is designed with twin screws, preferably counter-rotating screws, for the task of conveying, blending, compressing, heating and softening the blend. Depending on the choice of the components of the blend and the extrusion conditions, it may be that the blend will melt as well as soften. The screws which perform a significant part of this extrusion process are built of different smaller elements chosen from a variety of screw elements and kneader elements. Mixing and kneading time can be significantly altered by changing the type, length and configuration of the screw elements and possibly kneader elements. Short residence times and moderate to low shear forces contribute to safe processing and stable product even with heat sensitive drugs. Examples of available extruders include those manufactured by Leistritz, Brabender, Randcastle, and Kurimoto Co. Ltd.

Screw rotating speeds may play a part in the quality of the multiparticulates produced. High rotation speeds without appropriate compensation of the blend feed rate may produce high porosity multiparticulates with a variable drug release rate. On the other hand slow screw rotation would induce unnecessary long residence times. A vacuum connected to the extruder barrel is desirable to remove trapped air within the softened blend and thus produce dense non-porous multiparticulates.

The extrusion head is typically designed to produce multiple strands of fixed diameter. The number, shape and diameter of the orifices can be changed to suit a predetermined specification.

In addition to the screw speed, the other main influential parameters are the screw torque, individual barrel temperature, and extrusion head pressure and temperature.

In accordance with one cutting procedure of this invention, the extruded strands are carried away from the die-head on a conveyer. The strand diameter is affected by the blend feed rate, die-head orifice diameter, screw speed, barrel temperature, nip rolls speed and conveying speed. Conveying is appropriate to carry the extruded strand to a laser gauge or other measuring device to achieve a desired diameter such as 1.0 mm. During this conveying process the strands cool down gradually, but essentially remain flexible. Flexible strands retain integrity on the laser gauging device, between the pelletiser feed nip rolls and during entry to the pelletiser. Rapidly cooled strands, depending on the formulation, may lose their integrity and shatter during passage through the nip rolls and pelletiser into uneven-shaped and irregular-sized multiparticulates.

The strands are fed into the pelletiser by nip rolls. The pelletiser cuts the fed strands, for instance using a rotary knife cutter, to a pre-determined length, for example 1.0 mm. The feeding rate of the strands and the pelletiser cutter speed determine the length of the multiparticulates.

Overall, the co-ordination/interaction between the powder feeder, extruder, conveyor, laser gauge and pelletiser is an important parameter affecting the quantity, quality and reproducibility of the final multiparticulate products.

Multiparticulates produced by this cutting procedure where the extruded strands are carried away from the die-head typically take the form of cylinders.

In another preferred cutting procedure, a cutter cuts the extruded mix as it emerges under pressure and still softened from the orifices of the die plate. The cutter is suitably a rotary cutter with one or more blades which sweep over the surface of the die-head to pass the orifices. Two diametrically opposed blades are preferred. Ideally, the inner and outer surface boundaries to the extrusion orifices are coated with a non-stick material, e.g. a polytetrafluoroethylene (PIFE). As the cut extrudate particles expand and cool, they tend to form rounded surfaces. By appropriate adjustment of the extrusion pressure, the rate of extrusion and the speed of the cutter blade, it is possible to arrange for spherical or near-spherical multiparticulates to be obtained. Alternatively, this process can be operated to produce rods if desired. In one embodiment a stream of air is directed at the surface of the die-head, the air being at a reduced temperature to cool the extrudate and speed solidification.

Spherical multiparticulates produced by this method offer a number of possible advantages:

Better batch to batch reproducibility.

Easier coating and lower coating weight required.

Better capsule filling and higher yield.

More stable at elevated temperature.

More tamper resistant.

Reduced downstream processing.

Reduce or eliminate some problems that arise during conveying and pelletising the strands such as strands shattering to different length pellets and static charge.

The multiparticulates may be divided into unit doses such that each individual unit dose includes a dose of oxycodone sufficient to provide analgesia to a mammal, preferably a human patient. A suitable dose of oxycodone is 5 to 400 mg, especially 5 mg, 10 mg, 20 mg, 40 mg, 80 mg or 160 mg unit dosages. In this respect, a unit dose contains an effective amount of the therapeutically active agent to produce pain relief and/or analgesia to the patient. The dose of oxycodone administered to a patient will vary due to numerous factors, including the weight of the patient, the severity of the pain, the metabolic status and the nature of any other therapeutic agents being administered.

In one preferred embodiment, the multiparticulates are filled into hard gelatin capsules each containing a unit dose. The fill weight in the capsule is preferably in the range 80 to 500 mg, more preferably 120 to 500 mg. In a variation of this invention, the unit doses of multiparticulates may be incorporated into other solid pharmaceutical dosage formulations, for example using compression or shaping into tablets, or by forming the extruded product into the form of a suppository.

The capsules or other unit dose forms of this invention preferably are designed for administration at intervals of about 12 hours. To this end, the unit dose form suitably has an oxycodone dissolution rate in vitro, when measured by the USP Paddle Method (see the U.S. Pharmacopoeia XXII 1990) at 100 rpm in 900 ml aqueous buffer (pH between 1.6 and 7.2) at 37° C. of between 12.5 and 42.5% (by wt) oxycodone released after 1 hour, between 25 and 56% (by wt) oxycodone released after 2 hours, between 45 and 75% (by wt) oxycodone released after 4 hours and between 55 and 85% (by wt) oxycodone released after 6 hours. Furthermore, we prefer that the peak plasma level of oxycodone obtained in vivo occurs between 2 and 4.5 hours after administration of the dosage form.

More information on desirable characteristics for such oxycodone formulations is given in WO 9310765 which is incorporated herein in full by specific reference.

Using our specified method at pH 1.2, simulated gastric fluid, the release rates are suitably as follows:

| | | |
|---|---|---|
| Hour | % Released Lower Limit | % Released Upper Limit |

Preferred Limits

| | | |
|---|---|---|
| 1 | 16 | 56 |
| 2 | 37 | 77 |
| 4 | 60 | 100 |
| 10 | 75 | 100 |

More Preferable Limits

| | | |
|---|---|---|
| 1 | 21 | 51 |
| 2 | 42 | 72 |
| 4 | 65 | 95 |
| 10 | 80 | 100 |

Most Preferred Limits

| | | |
|---|---|---|
| 1 | 24 | 48 |
| 2 | 45 | 69 |
| 4 | 68 | 92 |
| 10 | 83 | 100 |

Using our specified method at pH 6.8, simulated intestinal fluid, the release rates are suitably as follows:

| | | |
|---|---|---|
| Hour | % Released Lower Limit | % Released Upper Limit |

Preferred Limits

| | | |
|---|---|---|
| 1 | 11 | 51 |
| 2 | 28 | 68 |
| 4 | 48 | 88 |
| 10 | 61 | 100 |

More Preferable Limits

| | | |
|---|---|---|
| 1 | 16 | 46 |
| 2 | 33 | 63 |
| 4 | 53 | 83 |
| 10 | 66 | 96 |

Most Preferred Limits

| | | |
|---|---|---|
| 1 | 19 | 43 |
| 2 | 36 | 60 |
| 4 | 56 | 80 |
| 10 | 69 | 93 |

As an alternative to administration at intervals of about 12 hours, the capsules or other unit dose forms of this invention are designed for administration at intervals of about 24 hours. To this end, the unit dose form suitably has an oxycodone dissolution rate in vitro, when measured by the USP Basket Method at 100 rpm in 900 ml aqueous buffer at a pH between 1.6 and 7.2 at 37° C. of from 0% to about 40% at 1 hour, from about 8% to about 70% at 4 hours, from about 20% to about 80% at 8 hours, from about 30% to about 95% at 12 hours, from about 35% to about 95% at 18 hours, and greater than about 50% at 24 hours. Furthermore, we prefer that the peak plasma level of oxycodone obtained in vivo is reached at about 2 hours to about 17 hours after administration at steady state of the dosage form.

More information on desirable characteristics for such oxycodone formulations is given in WO 02087512 which is incorporated herein in full by specific reference.

In a variation, the present invention provides unit doses which contain oxycodone and an oxycodone antagonist effective to prevent tampering. In this respect, reference is made to WO 0313433 which is incorporated herein in full by specific reference. In particular, the unit dose can contain oxycodone and naltrexone. Other opioid antagonists which are known in the art can be used, for example naloxone.

The present invention provides extruded multiparticulates of oxycodone, and extruded multiparticulates of oxycodone antagonist such as naltrexone. The naltrexone multiparticulates do not release naltrexone on conventional administration, and for example have a non-release coating. Both populations are preferably visually and physically identical.

An important aspect of this invention is a capsule with a unit dose fill of less than 500 mg, comprising up to about 350 mg of oxycodone multiparticulates, and up to about 200 mg of tamper-proof oxycodone antagonist multiparticulates. For example, there can be 120 to 300 mg of oxycodone multiparticulates, and 125 to 175 mg of tamper-proof oxycodone antagonist multiparticulates.

### SUMMARY OF THE DRAWINGS

Reference is made in the following experimental section to the accompanying drawings, in which:

FIG. 1 is a schematic representation of one of the screw trains of the Leistritz 18 twin screw extruder used in the Examples.

FIG. 2 shows the effect of the stearyl alcohol:stearic acid ratio on the release rate of oxycodone extrusion multiparticulates.

FIG. 3 shows the effect of Eudragit RL PO on the release rate of oxycodone hydrochloride from extruded multiparticulates containing 8.3% w/w oxycodone.

FIG. 4 shows the effect of Eudragit RL PO on the release rate of oxycodone hydrochloride from extruded multiparticulates containing 25% w/w oxycodone.

FIG. 5 shows the effect of microcrystalline cellulose on the release rate of oxycodone hydrochloride from extruded multiparticulates containing 8.3% w/w oxycodone.

FIG. 6 shows the effect of microcrystalline cellulose on the release rate of oxycodone hydrochloride from extruded multiparticulates containing 25% w/w oxycodone.

FIG. 7 shows the effect of high viscosity HPMC on the release rate of oxycodone hydrochloride from extruded multiparticulates containing 8.3% w/w oxycodone.

FIG. 8 shows the effect of high viscosity HPMC on the release rate of oxycodone hydrochloride from extruded multiparticulates containing 25% w/w oxycodone.

FIG. 9 provides some in vitro dissolution data for three batches of multiparticulates of this invention and for the commercial product OxyContin® Tablets.

FIGS. 10 to 16 provide in vivo data for the three batches of FIG. 9 and for the commercial product OxyContin® Tablets.

FIGS. 17 to 19 give some further in vitro dissolution curves.

FIG. 20 provides a comparison of dissolution profiles of capsules of Example 22 with other products.

FIG. 21 provides a comparison of dissolution profiles of 40 mg oxycodone q12 hr capsules of Examples 24 and 25.

### EXAMPLES OF THE INVENTION

Standardised Conditions

For the following experimental work, standardised conditions were established for the extrusion of oxycodone hydrochloride blends. The extruder was a Leistritz 18 at 140 rpm, with a feed rate of 2.6 kg/h producing pellets of 1 mm diameter and 1 mm length.

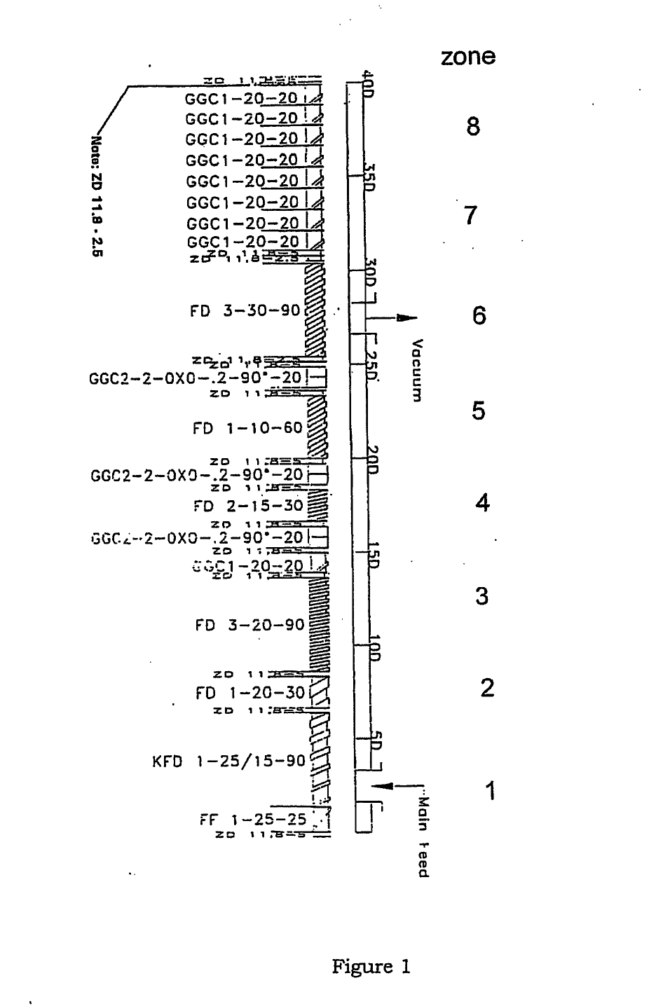

The design of the screw is shown in FIG. 1 using components indicated by the manufacturing codes of the distributor Leistritz USA. The aim is to optimise the mixture by adding extra mixing elements ‘GGC2’ or ‘ZS’ to avoid mixing problems, and to increase the residence time by including ‘FD’ elements to avoid wetting problems.

The extruder comprises ten zones, with zone 1 extending from 0 to 5D on FIG. 1; zone 2 extending from 5D to 10D on FIG. 1, and so on up to zone 8 extending from 35D to 40D, and then zones 9 and 10 are at the extruder head.

Typical batch zone temperatures were as follows (° C.):

| | | | | | | | Melt pressure | Torque |
|---|---|---|---|---|---|---|---|---|
| Example | 1 | 2 | 3-6 | 7-8 | 9 | 10 | (bar) | (%) |
| 5 | 14 | 40 | 90 | 75 | 85 | 90 | 63-68 | 53-59 |
| 8 | 14 | 40 | 90 | 75 | 85 | 90 | 61-62 | 49 |
| 9 | 14 | 40 | 125 | 120 | 125 | 125 | 99-107 | 78-84 |
| 10 | 14 | 40 | 120 | 105-106 | 115 | 120 | 73-77 | 74-79 |
| 11 | 14 | 40 | 101-103 | 100 | 106 | 106 | 99-115 | 89-97 |

For Examples 9 to 11, the temperatures were raised significantly. The feed rate and screw speed were generally kept constant although the conveyor speed, nip rolls speed and pelletiser speed changed according to the properties of the extrudate when it emerged from the die plate (this was highly dependent on the way the extrudate expanded and hence hard to correlate to previous batches).

Two drug loads (8.3 and 25% by weight) of oxycodone extruded multiparticulate formulations (see tables) were planned to cover doses of 10 mg and 40 mg.

For the 8.3% oxycodone load, the following trial batches were prepared, where the weights are mg per unit dose.

| | Example | | | |
|---|---|---|---|---|
| | 1 (Comparative) | 2 | 3 | 4 |
| Oxycodone HCl | 10 | 10 | 10 | 10 |
| Eudragit RS PO | 77 | 72 | 62 | 74 |
| Stearyl alcohol | 24.75 | 24 | 24 | 24 |
| Stearic acid | 8.25 | 4 | 4 | 4 |
| Microcrystalline | | 10 | | |
| cellulose (Avicel PH101) | | | | |
| Eugragit RL PO | | | 20 | 8 |
| Hydroxypropylmethyl | | | | |
| cellulose (HPMC K100M) | | | | |
| Total | 120 | 120 | 120 | 120 |

Example

| | 5 | 6 | 7 | 8 |
|---|---|---|---|---|
| Oxycodone HCl | 10 | 10 | 10 | 10 |
| Eudragit RS PO | 77 | 69 | 74 | 70 |
| Stearyl alcohol | 24 | 24 | 16 | 16 |
| Stearic acid | 4 | 4 | 12 | 12 |
| Microcrystalline | | 13 | | |
| cellulose (Avicel PH101) | | | | |
| Eugragit RL PO | 5 | | | |
| Hydroxypropylmethyl | | | 8 | 12 |
| cellulose (HPMC K100M) | | | | |
| Total | 120 | 120 | 120 | 120 |

| | Example | | |
|---|---|---|---|
| | 9 | 10 | 11 |
| Oxycodone HCl | 10 | 10 | 10 |
| Eudragit RS PO | 68 | 66 | 74 |
| Stearyl alcohol | 8 | 14 | 14 |
| Eudragit RL PO | 28 | 25 | 17 |
| Glyceryl behenate | 6 | 5 | 5 |
| Total | 120 | 120 | 120 |

For the 25% oxycodone load, the following trial batches were prepared, where the weights are mg per unit dose.

| | Example | | | | |
|---|---|---|---|---|---|
| | 12 | 13 | | | |
| | Comparative | Comparative | 14 | 15 | 16 |
| Oxycodone HCl | 40 | 40 | 40 | 40 | 40 |
| Eudragit RS PO | 90 | 90 | 85 | 87 | 82 |
| Stearyl alcohol | 10 | 20 | 20 | 20 | 20 |
| Stearic acid | 20 | 10 | 10 | 10 | 10 |
| Eugragit RL PO | | | 5 | 3 | 8 |
| Total | 160 | 160 | 160 | 160 | 160 |

| | Example | | |
|---|---|---|---|
| | 17 | 18 | 19 |
| Oxycodone HCl | 40 | 40 | 40 |
| Eudragit RS PO | 78 | 82 | 78 |
| Stearyl alcohol | 20 | 8 | 8 |
| Stearic acid | 10 | 22 | 22 |
| Microcrystalline | 12 | | |
| cellulose (Avicel PH101) | | | |
| Hydroxypropylmethyl | | 8 | 12 |
| cellulose (HPMC K100M) | | | |
| Total | 160 | 160 | 160 |

Release Rate Studies

The oxycodone extruded multiparticulates of Examples 1 to 19 were tested for dissolution using Ph.Eur. basket dissolution apparatus at 37° C., 100 rpm in 900 ml of USP simulated gastric fluid at pH 1.2 without enzyme. Standard HPLC procedures were used for assay.

Additionally, the oxycodone extruded multiparticulates of Example 9 were tested for dissolution using Ph.Eur. basket dissolution apparatus at 37° C., 100 rpm in 900 ml of simulated intestinal fluid at pH 6.8 without enzyme. Again, standard HPLC procedures were used for assay.

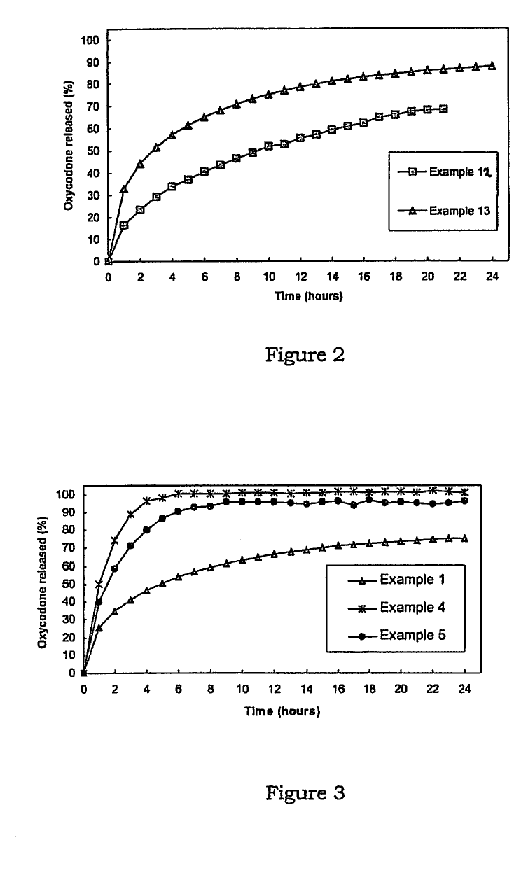

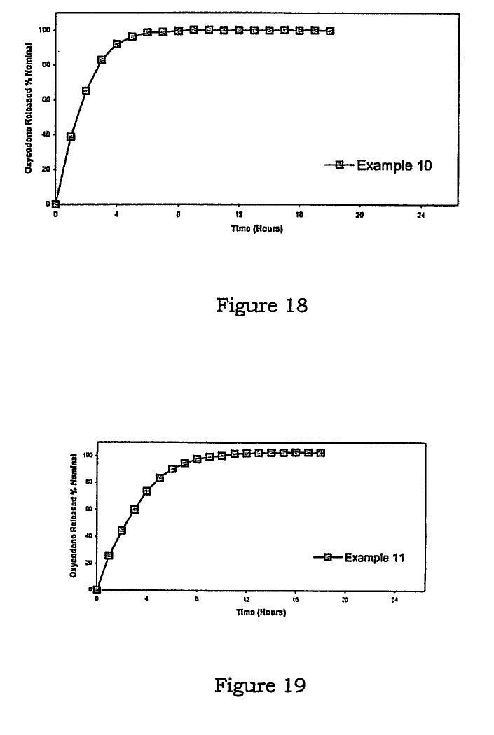

The in vitro release rates were measured, and gave the results plotted in the accompanying FIGS. 2 to 9 and 17 to 19.

Eudragit RL PO

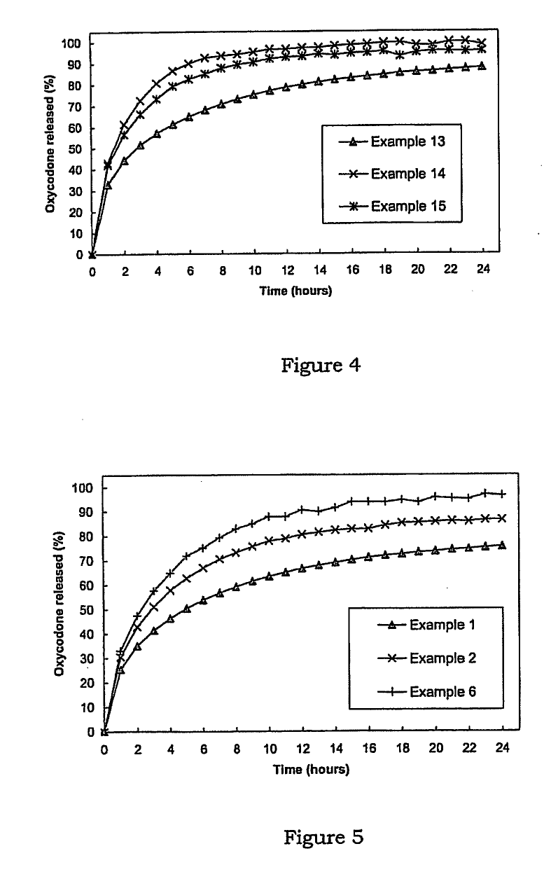

With the load of 8.3% oxycodone hydrochloride, the presence in the extruded multiparticulates of 5, 8 or 20 mg Eudragit RL PO/120 mg significantly enhanced the release rate (see FIG. 3). Similarly, with the 25% oxycodone loaded multiparticulates, 3 and 5 mg Eudragit RL PO/160 mg showed a comparable effect on the release rate (see FIG. 4).

Microcrystalline Cellulose

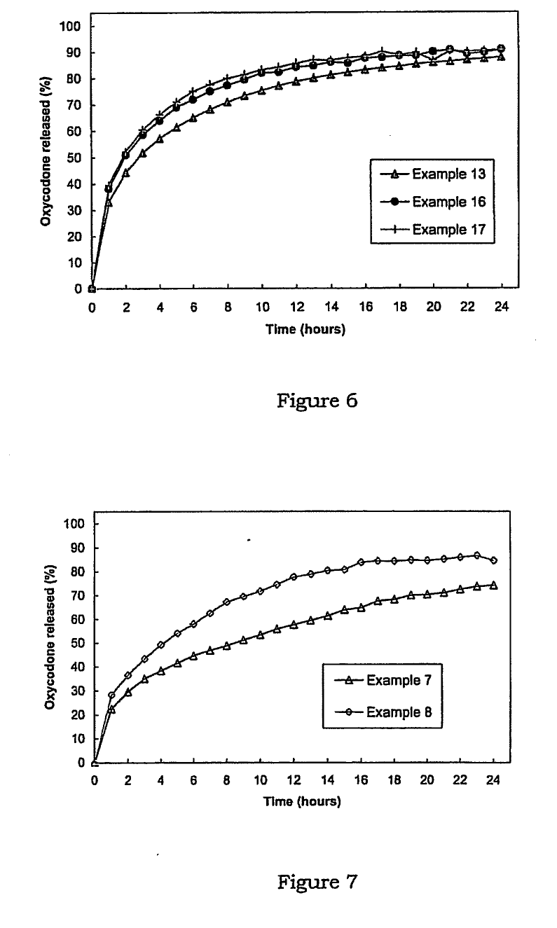

10 and 13 mg/120 mg oxycodone extruded multiparticulates and 8 and 12 mg/160 mg oxycodone extruded multiparticulates were used in the 8.3% and 25% oxycodone hydrochloride loaded formulations respectively. The effect of the microcrystalline cellulose on the release rate and total release of oxycodone hydrochloride is presented in FIGS. 5 and 6 for 8.3% and 25% drug load, respectively.

Hydroxypropyl Methylcellulose

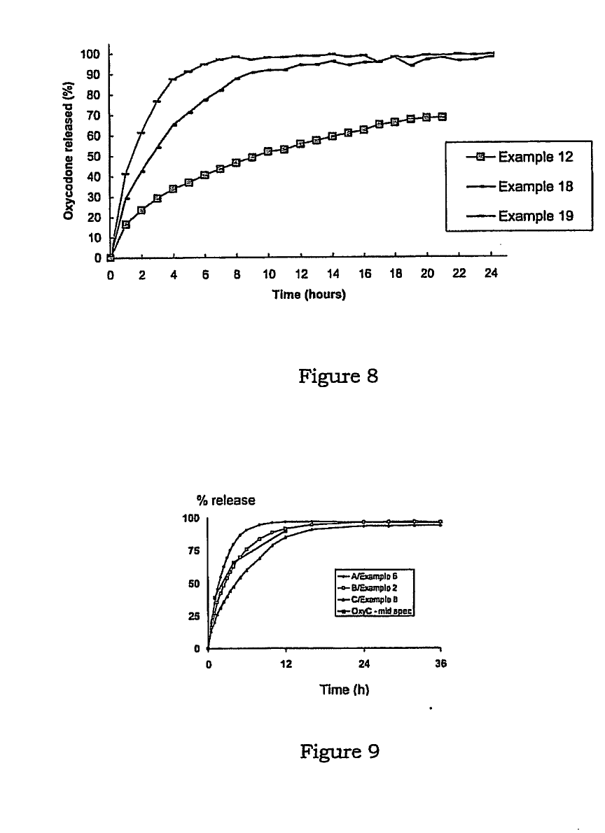

High viscosity HPMC (HPMC K100M) at levels of 8 and 12 mg/120 mg and 8 and 12 mg/160 mg were employed for 8.3% and 25% drug load extruded multiparticulates respectively. The dissolution release study indicates that more pronounced total release of oxycodone hydrochloride was achieved at later time points (see FIGS. 7 and 8).

Glyceryl Behenate

Dissolution data for the formulations of Examples 9 to 11 is given in FIGS. 17 to 19, and demonstrates that the inclusion of glyceryl behenate can give the desired high initial release combined with high total release. In FIG. 17, SGF indicates results for simulated gastric fluid, and SIF indicates results for simulated intestinal fluid. It can be seen that the release of oxycodone is substantially independent of pH.

The currently preferred products are Examples 9, 10 and 11, with Examples 10 and 11 being most preferred.

Bioavailability Study

The formulations of Examples 2, 5 and 8 were investigated along with OxyContin® Tablets in a Phase I bioavailability study, where they were identified respectively as B, A and C. The study was a four-period randomised incomplete block crossover study, involving 24 healthy male and female subjects. A single dose of 2×10 mg capsules (20 mg total) of Example 2, Example 5, Example 8 or a 20 mg OxyContin® Tablet was administered to the subjects. Each test formulation was administered after an overnight fast, or following ingestion of a high fat breakfast.

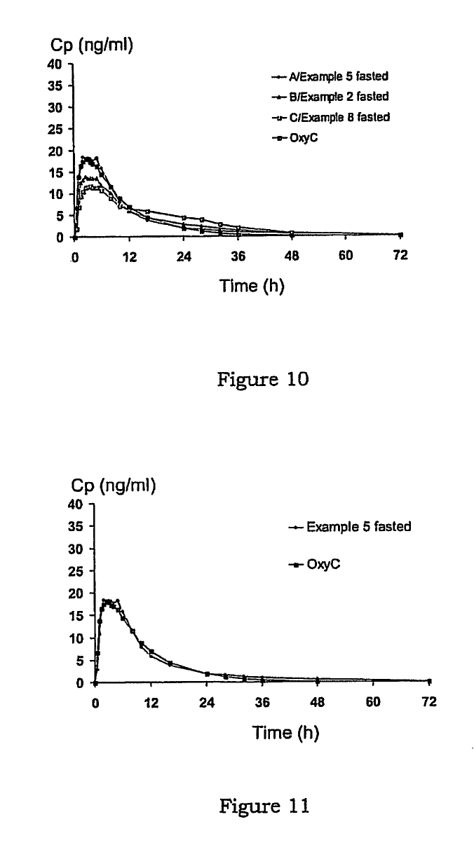

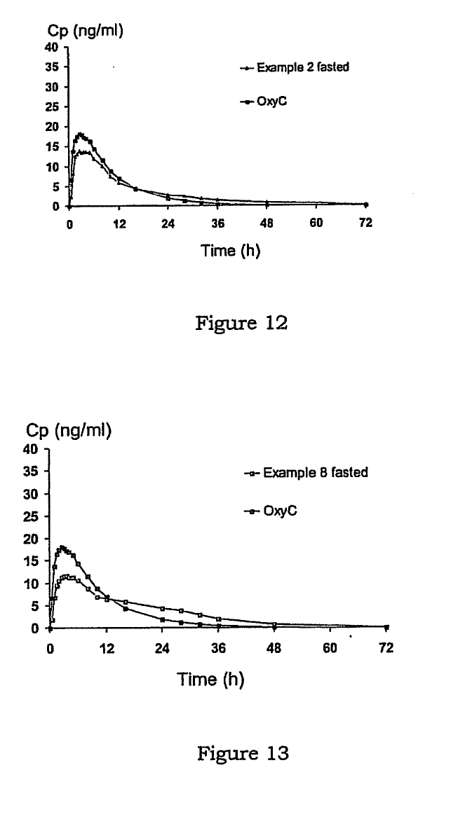

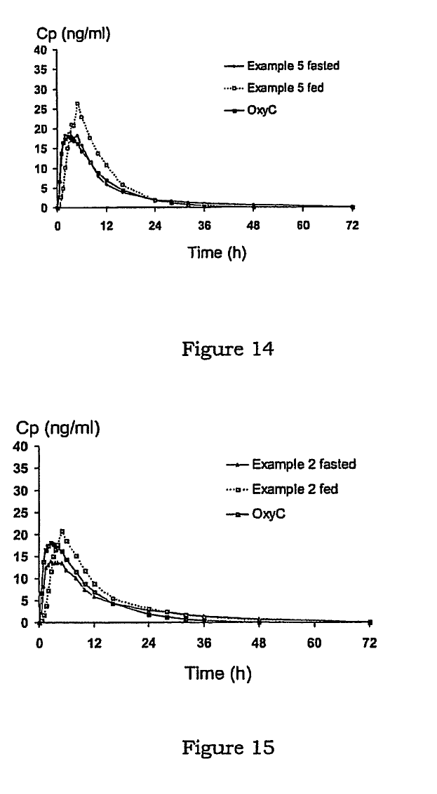

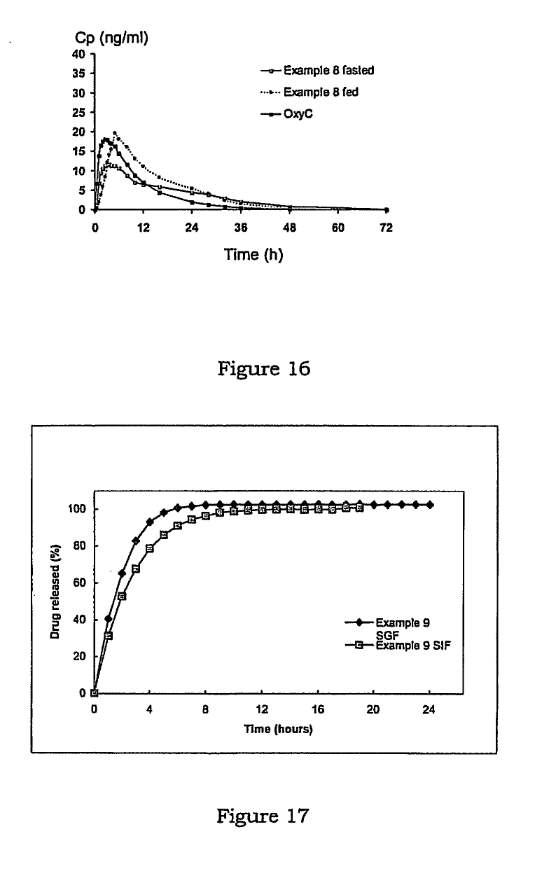

The mean in vivo plasma profiles from this study are illustrated in FIGS. 10 to 16, and the mean parameters are summarised in the following table. The in vitro dissolution data for these formulations and for OxyContin® Tablets is shown in FIG. 9.

| | Example 5 | Example 5 | Example 2 | Example 2 |
|---|---|---|---|---|
| | fasted | fed | fasted | fed |
| | (n = 13) | (n = 13) | (n = 11) | (n = 14) |
| AUCt | 223.2 | 272.4 | 212.2 | 255.5 |
| (ng · h/mL)* | | | | |
| SD | (47.07) | (76.93) | (48.49) | (44.91) |
| AUC_{INF} | 231.9 | 277.7 | 220.3 | 261.3 |
| (ng · h/mL)* | | | | |
| SD | (46.16) | (77.27) | (51.54) | (45.83) |
| Cₘₐₓ | 21.6 | 26.9 | 15.4 | 21.5 |
| (ng/mL)* | | | | |
| SD | (5.07) | (6.78) | (2.81) | (4.12) |
| tₘₐₓ (h)** | 3.0 | 5 | 3 | 5 |
| Range | (2-6) | (2.5-5) | (2-5) | (3-6) |

*arithmetic mean
**median

| | | | OxyContin ® |
|---|---|---|---|
| | Example 8 | Example 8 | Tablets |
| | fasted (n = 14) | fed (n = 12) | (n = 13) |
| AUCt (ng · h/mL)* | 232.9 | 298.19 | 210.6 |
| SD | (45.32) | (51.63) | (33.07) |
| AUC_{INF} (ng · h/mL)* | 239.6 | 302.3 | 212.6 |
| SD | (44.90) | (53.63) | (32.76) |
| Cₘₐₓ (ng/mL)* | 12.4 | 20.0 | 19.1 |
| SD | (3.52) | (3.73) | (4.34) |
| tₘₐₓ (h)** | 3.5 | 5 | 2.5 |
| Range | (2-6) | (5-8) | (1.5-5) |

*arithmetic mean
**median

With the exception of Example 8, the oxycodone formulations provided an equivalent bioavailability of oxycodone in terms of AUCₜ and AUC_{INF}, relative to OxyContin® Tablets and relative to each other. FIG. 10 shows that all three formulations have similar mean plasma oxycodone concentrations at 12 hours, suggesting that all three formulations show potential for being developed as a 12 hourly product. FIG. 11 shows that Example 5 fasting was most similar to OxyContin® Tablets in terms of AUCₜ, AUC_{INF} and Cₘₐₓ.

### Examples 20 and 21

Q12 Hr formulations were prepared with a drug load of 30.3% w/w, to enable filling into size 1 capsules: 40 mg in 132 mg dose weight and 80 mg in 264 mg dose weight. The component levels enabled relatively low processing temperatures to be achieved. The conveyor and pelletiser speeds were optimised during processing. The processing conditions for Example 21 are shown. Further improvements in processing conditions, i.e., melt pressure and screw torque, were obtained after adjustment of the extrusion die plate depth from 3.7 mm to 2.4 mm.

| Quantity (mg) per unit dose weight | | |
|---|---|---|
| (% of total) | | |
| | Example 20 | Example 21A, 21B |

| | | | | |
|---|---|---|---|---|
| Oxycodone HCl | 40.0 | (30.3%) | 40.0 | (30.3%) |
| Eudragit RSPO | 64.0 | (48.5%) | 62.0 | (47.0%) |
| Eudragit RLPO | 10.0 | (7.6%) | 9.0 | (6.8%) |
| Stearyl alcohol | 12.0 | (9.1%) | 15.0 | (11.4%) |
| Glycerol dibehenate | 6.0 | (4.5%) | 6.0 | (4.5%) |
| Total | 132 | mg | 132 | mg |

Extruder Processing Conditions:

| | |
|---|---|
| Extruder: | Leistritz Micro 18 |
| Screw configuration: | See diagram in FIG. 1 |
| Feed rate (kg/hour): | 2.6 |
| Screw speed (rpm): | 140 |
| Die plate orifice diameter (mm): | 1.0 (8 orifice plate) |
| Pellet dimensions: | 1.0 mm × 1.0 mm (range 0.8-1.2 mm) |

### Examples 21A

| | | | | | |
|---|---|---|---|---|---|
| Heating zone: | 1 | 2 | 3-6 | 7-8 | 9-10 |
| Temp* (° C. ) | 14 | 40 | 102-103 | 103 | 104 |

Torque (%): 81-84
Melt Pressure(bar): 79-93
Die plate orifice depth (mm): 3.7

### Example 21B

| | | | | | |
|---|---|---|---|---|---|
| Heating zone: | 1 | 2 | 3-6 | 7-8 | 9-10 |
| Temp* (° C. ) | 14 | 40 | 102-103 | 102-103 | 104 |

Torque (%): 74-76
Melt Pressure(bar): 70-73
Die plate orifice depth (mm): 2.4

### Example 22

A formulation was prepared based on Example 21 with further adjusted plasticiser/lubricant components. Processing was carried out using an extrusion die plate with an orifice depth of 2.4 mm. The temperature and die plate conditions used were as reported for Example 21B.

| | Quantity (mg) per unit | |
|---|---|---|
| | dose weight (% of total) | |
| | Example 22 | |
| Oxycodone HCl | 40.0 | (30.3%) |
| Eudragit RSPO | 66.0 | (50.0%) |
| Eudragit RLPO | 6.0 | (4.5%) |
| Stearyl alcohol | 14.0 | (10.6%) |
| Glycerol dibehenate | 6.0 | (4.5%) |
| Total | 132 | mg |

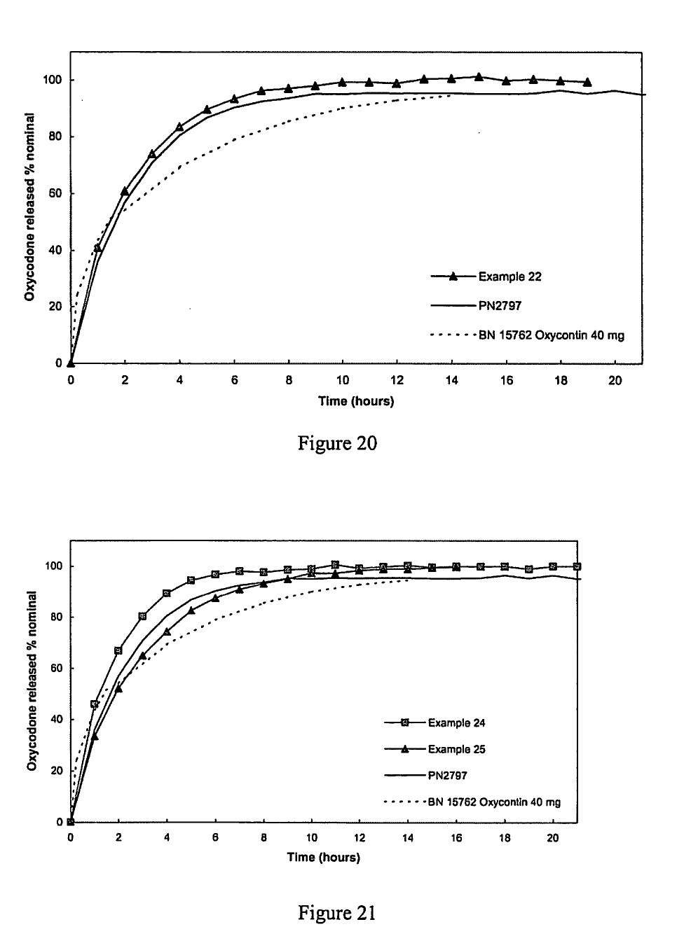

Dissolution tests were carried out for the capsules of Example 22, also referred to by batch number F764/67. As shown in FIG. 20, the oxycodone dissolution profile compared well with the target profile designated PN2797 (encapsulated product). The profile for a commercial batch of OxyContin® 40 mg tablets is also given in FIG. 20.

### Example 23

A further formulation with a reduced content of stearyl alcohol was designed to ensure improved stability to storage and minimise changes in the dissolution profiles during storage. This approach had previously been shown to improve the stability of the dissolution rate under accelerated storage conditions for 10/20 mg formulations.

Acceptable extrusion processing conditions could not be established on the Micro 18 extruder due to the maximum torque limit being reached with these formulations. These formulations would, however, be recommended for processing on a Micro 27 extruder, which is able to handle higher torque levels, to generate products with improved storage stability.

| | Quantity (mg) per unit | |
|---|---|---|
| | dose weight (% of total) | |
| | Example 23 | |
| Oxycodone HCl | 40.0 | (30.3%) |
| Eudragit RSPO | 67.0 | (50.8%) |
| Eudragit RLPO | 7.0 | (5.3%) |
| Stearyl alcohol | 12.0 | (9.1%) |
| Glycerol dibehenate | 6.0 | (4.5%) |
| Total | 132 | mg |

### Examples 24 and 25

As a result of these findings, two formulations including the lubricant glycerol dibehenate were proposed, although the processing conditions for these formulations are at the limits of the torque capability of the Micro 18.

| Quantity (mg) per unit | | |
|---|---|---|
| dose weight (% of total) | | |
| | Example 24 | Example 25 |

| | | | | |
|---|---|---|---|---|
| Oxycodone HCl | 40.0 | (30.3%) | 40.0 | (30.3%) |
| Eudragit RSPO | 63.0 | (47.7%) | 69.0 | (52.3%) |
| Eudragit RLPO | 9.0 | (6.8%) | 3.0 | (2.3%) |
| Stearyl alcohol | 14.0 | (10.6%) | 14.0 | (10.6%) |
| Glycerol dibehenate | 6.0 | (4.5%) | 6.0 | (4.5%) |
| Total | 132 | mg | 132 | mg |

The processing conditions used are given.

| | |
|---|---|
| Extruder: | Leistritz Micro 18 |
| Screw configuration: | See FIG. 1 |

| | | | | | | |
|---|---|---|---|---|---|---|
| Heating zone: | 1 | 2 | 3-6 | 7-8 | 9 | 10 |
| Temp* (° C. ) | 14 | 40 | 103 | 102 | 103 | 103 |

Torque (%): 81-90
Melt Pressure(bar): 81-95
Feed rate (kg/hour): 2.6
Screw speed (rpm): 140
Die plate orifice diameter (mm): 1.0 (8 orifice plate)
Die plate orifice depth (mm): 2.4
Pellet dimensions: 1.0 mm × 1.0 mm (range 0.8-1.2 mm)

To facilitate provision of the required dose, MEMs were filled as a 40 mg strength using size 1 capsules and placed on a formal stability programme.

Dissolution tests were carried out for the capsules of Examples 24 and 25, also referred to by batch numbers F767/75 and F769/22, respectively. The dissolution profiles for Examples 24 and 25 and comparable batches are given in FIG. 21.

### Example 26

### A Combination Tamper Resistant Product

Co-encapsulation of extruded oxycodone multiparticulates and extruded naltrexone or naloxone multiparticulates can be used for a tamper resistant combination product.

Oxycodone multiparticulates and naltrexone multiparticulates as described in WO 03013433 may be filled into capsules using a single or dual stage filling process. The quantity of naltrexone multiparticulates which may be filled is 150 mg, containing 8 mg of naltrexone. The recommended fill weights of oxycodone multiparticulates to achieve oxycodone doses ranging from 10 mg to 40 mg are as follows (see also the following table):
1. 120 mg and 240 mg of 8.3% (w/w) drug loaded multiparticulates for oxycodone doses of 10 mg and 20 mg, respectively.
2a. 120 mg of 33.3% (w/w) drug loaded multiparticulates for an oxycodone dose of 40 mg or
2b. 160 mg of 25% (w/w) drug loaded multiparticulates for an oxycodone dose of 40 mg.

In addition, 5 mg and 80 mg oxycodone doses may also be considered, with respective capsule fill weights as follows:
1. 60 mg of 8.3% (w/w) drug loaded multiparticulates for an oxycodone dose of 5 mg.
2a. 240 mg of 33.3% (w/w) drug loaded multiparticulates for an oxycodone dose of 80 mg or
2b. 320 mg of 25% (w/w) drug loaded multiparticulates for an oxycodone dose of 80 mg.

For the drug load of 33.3% (w/w), the following trial formulations indicated 26.A and 26.B were prepared, where the weights are mg per unit dose:

| | 26.A | 26.B |
|---|---|---|
| Oxycodone HCl | 40.0 | 40.0 |
| Eudragit RS PO | 67.0 | 67.0 |
| Stearyl Alcohol | 13.0 | 8.0 |
| Glyceryl behenate | | 5.0 |
| Total | 120 | 120 |

These two formulations were initially manufactured for proof of principle for a higher strength product, and without Eudragit RL PO. The dissolution profiles from these formulations were slower than required and can be readily modified by the use of a water permeability modifier in accordance with the invention.

Capsule filling of the required proportions of oxycodone and naltrexone multiparticulates may be achieved using either a single stage process or preferably a dual stage filling process. In the single stage filling process, the respective proportions of multiparticulates may be pre-blended and filled into capsules either by manual or preferably automated process. By the preferred dual stage filling process, one type of multiparticulates can be filled in a first stage, either by manual or preferably automated processes. The second type of multiparticulates can then be filled in the second filling stage, again either by manual or preferably automated processes.

The theoretical fill weights for a range of capsule strengths based on drug loading are given in the following tables.

oxycodone loading 8.3% w/w

| | | oxycodone and | |
|---|---|---|---|
| oxycodone mg | oxycodone multi- | naltrexoneØ multi- | |
| per capsule | particulates (mg) | particulates (mg) | |
| 10 | 120 | 270 | (capsule Size 1) |
| 20 | 240 | 390 | (capsule Size 0) |
| 40 | 480 | 630 | (can not be filled) |
| 5+ | 60* | 210 | (capsule Size 1) |
| 80+ | 960 | 1110 | (can not be filled) |

*Weight below assumed minimum possible capsule fill weight.
+Included as an illustration of possibilities, if lower or higher strengths in the range are required.
Ø120 mg naltrexone multiparticulates + 20% coat.

oxycodone loading 25% w/w

| | | oxycodone and |
|---|---|---|
| Oxycodone mg | oxycodone multi- | naltrexoneØ multi- |
| per capsule | particulates (mg) | particulates (mg) |
| 10 | 40* | Low to fill |
| 20 | 80 | 230 (capsule Size 1) |
| 40 | 160 | 310 (capsule Size 0) |
| 5+ | 20* | Low to fill |
| 80+ | 320 | 470 (capsule Size 0E) |

*Weight below assumed minimum possible capsule fill weight.
+Included as an illustration of possibilities, if lower or higher strengths in the range are required.
Ø120 mg naltrexone multiparticulates + 20% coat.

### Example 27

### Alternate Cutter Procedure

For this Example, an alternate cutting procedure was employed. Extrudate emerges from the twelve orifices of the die-head shown in FIG. 8 of a Leistritz 18 extruder. A rotary cutter with two blades is used to cut the extruded mix as it emerges under pressure and still molten from the orifices of the die plate. The blades sweep over the surface of the die-head to pass the orifices. As they expand and cool, the cut extrudate particles tend to form rounded surfaces.

The following formulation was employed.

| Material | % w/w |
|---|---|
| Lactose anhydrous | 10.0 |
| Eudragit RS PO | 91.0 |
| Triethyl citrate | 10.0 |
| PEG 6000 | 6.0 |
| Magnesium Stearate | 4.5 |
| Total | 121.5 |

By appropriate adjustment of the extrusion parameters, including temperatures and rates of cooling, spherical or substantially spherical multiparticulates may be obtained.

1. Multiparticulates which contain oxycodone and have a high initial release of oxycodone, and a high total release of oxycodone.

2. Multiparticulates according to claim 1, which release at least 60% oxycodone after 4 hours, when tested by a specified test method which comprises using Ph.Eur. basket dissolution apparatus at 37° C., 100 rpm in 900 ml of USP simulated gastric fluid at pH 1.2 without enzyme.

3. Multiparticulates according to claim 2, which release at least 70% oxycodone after 4 hours, when tested by the specified test method.

4. Multiparticulates according to claim 3, which release at least 80% oxycodone after 4 hours, when tested by the specified test method.

5. Multiparticulates according to claim 4, which release 100% oxycodone after 12 hours, when tested by the specified test method.

6. Multiparticulates according to claim 4, which release 95% oxycodone after 10 hours, when tested by the specified test method.

7. Multiparticulates according to claim 6, which release at least 85% oxycodone after 8 hours, when tested by the specified test method.

8. Multiparticulates of oxycodone with some pharmacokinetic/pharmacodynamic properties which resemble OxyContin® Tablets.

9. Multiparticulates of oxycodone which include a water permeability modifier to allow preparation of a mimic for OxyContin® Tablets by extrusion.

10. Multiparticulates which contain (a) oxycodone, (b) water-insoluble ammonium methacrylate copolymer, (c) plasticiser, (d) lubricant and (e) water permeability modifier.

11. Multiparticulates according to claim 10, wherein the oxycodone is present as a pharmaceutically acceptable salt.

12. Multiparticulates according to claim 11, wherein the oxycodone is present as oxycodone hydrochloride.

13. Multiparticulates according to claim 10, wherein the plasticiser is chosen from cetyl alcohol, stearyl alcohol, cetostearyl alcohol, sorbitol, sucrose, high molecular weight polyethylene glycol, dibutyl sebacate, tributyl citrate, triethyl citrate, propylene glycol and low molecular weight polyethylene glycol.

14. Multiparticulates according to claim 13, wherein the plasticiser is stearyl alcohol.

15. Multiparticulates according to claim 13, wherein the plasticiser is a high molecular weight polyethylene glycol.

16. Multiparticulates according to claim 10, wherein the lubricant is chosen from glyceryl behenate, talc and silicone dioxide.

17. Multiparticulates according to claim 16, wherein the lubricant is glyceryl behenate.

18. Multiparticulates according to claim 10, wherein the lubricant is stearic acid or a stearate salt.

19. Multiparticulates according to claim 10, wherein the water permeability modifier is selected from an insoluble hydrophilic wicking agent, a gelling agent which hydrates to form a gel to control the water movement, a high molecular weight polyethylene glycol, or a water permeable ammonium methacrylate copolymer.

20. Multiparticulates according to claim 19, wherein the water permeability modifier is selected from microcrystalline cellulose, croscarmellose sodium, crospovidone, sodium starch glycollate, a high molecular weight hydrogel, a high viscosity poly(ethylene oxide), and a water permeable ammonium methacrylate copolymer.

21. Multiparticulates according to claim 20, wherein the water permeability modifier is a water permeable ammonium methacrylate copolymer.

22. Multiparticulates according to claim 10, wherein the percentage amounts of the ingredients (a) to (e) are as given in the following table, based on the total weight of the five ingredients:

| | |
|---|---|
| oxycodone as hydrochloride | 3 to 50 |
| insoluble ammonium methacrylate copolymer | 25 to 85 |
| plasticiser | 1 to 30 |
| lubricant | 1 to 25 |
| water permeability modifier | 1 to 40. |

23. Multiparticulates according to claim 22, wherein the percentage amounts of the ingredients (a) to (e) are as given in the following table, based on the total weight of the five ingredients:

| | |
|---|---|
| oxycodone as hydrochloride | 5 to 40 |
| insoluble ammonium methacrylate copolymer | 35 to 75 |
| plasticiser | 3 to 25 |
| lubricant | 2 to 25 |
| water permeability modifier | 1 to 30. |

24. Multiparticulates according to claim 23, wherein the percentage amounts of the ingredients (a) to (e) are as given in the following table, based on the total weight of the five ingredients:

| | |
|---|---|
| oxycodone as hydrochloride | 7.5 to 35 |
| insoluble ammonium methacrylate copolymer | 50 to 65 |
| plasticiser | 5 to 15 |
| lubricant | 2 to 25 |
| water permeability modifier | 1 to 20 |

25. Multiparticulates according to claim 10, which contain oxycodone, Eudragit RS PO, stearyl alcohol, glyceryl behenate, and Eudragit RL PO.

26. A pharmaceutical composition in unit dose form comprising multiparticulates according to claim 10.

27. A pharmaceutical composition according to claim 26, wherein the unit dose provides a dose of oxycodone sufficient to provide analgesia to a human patient.

28. A pharmaceutical composition according to claim 27 which is bioequivalent to OxyContin® Tablets in one or more respects.

29. A pharmaceutical composition according to claim 27, wherein the sufficient dose of oxycodone is 5 to 400 mg.

30. A pharmaceutical composition according to claim 29, wherein the unit dose of oxycodone is 5 mg, 10 mg, 20 mg, 40 mg, 80 mg or 160 mg.

31. A pharmaceutical composition according to claim 26, in the form of a capsule with a fill of said multiparticulates.

32. A pharmaceutical composition according to claim 31, wherein the multiparticulates are filled into hard gelatin capsules each containing a unit dose.

33. A pharmaceutical composition according to claim 32, wherein the fill weight in the range 120 to 500 mg.

34. A pharmaceutical composition according to claim 26, which is intended for administration at intervals of about 12 hours.

35. A pharmaceutical composition according to claim 34, wherein the unit dose form has an oxycodone dissolution rate in vitro, when measured by the USP Paddle Method (see the U.S. Pharmacopoeia XXII 1990) at 100 rpm in 900 ml aqueous buffer (pH between 1.6 and 7.2) at 37° C. of between 12.5 and 42.5% (by wt) oxycodone released after 1 hour, between 25 and 56% (by wt) oxycodone released after 2 hours, between 45 and 75% (by wt) oxycodone released after 4 hours and between 55 and 85% (by wt) oxycodone released after 6 hours.

36. A pharmaceutical composition according to claim 35, wherein the peak plasma level of oxycodone obtained in vivo occurs between 2 and 4.5 hours after administration.

37. A pharmaceutical composition according to claim 34, wherein the release rates of oxycodone meet the following lower and upper limits:

| Hour | % Released Lower Limit | % Released Upper Limit |
|---|---|---|
| 1 | 16 | 56 |
| 2 | 37 | 77 |
| 4 | 60 | 100 |
| 10 | 75 | 100 |

when tested by a specified test method which comprises using Ph.Eur. basket dissolution apparatus at 37° C., 100 rpm in 900 ml of USP simulated gastric fluid at pH 1.2 without enzyme.

38. A pharmaceutical composition according to claim 37, wherein the release rates of oxycodone meet the following lower and upper limits:

| Hour | % Released Lower Limit | % Released Upper Limit |
|---|---|---|
| 1 | 21 | 51 |
| 2 | 42 | 72 |
| 4 | 65 | 95 |
| 10 | 80 | 100 |

when tested by the specified test at pH 1.2.

39. A pharmaceutical composition according to claim 38, wherein the release rates of oxycodone meet the following lower and upper limits:

| Hour | % Released Lower Limit | % Released Upper Limit |
|---|---|---|
| 1 | 24 | 48 |
| 2 | 45 | 69 |
| 4 | 68 | 92 |
| 10 | 83 | 100 |

when tested by the specified test at pH 1.2.

40. A pharmaceutical composition according to claim 34, wherein the release rates of oxycodone meet the following lower and upper limits:

| Hour | % Released Lower Limit | % Released Upper Limit |
|---|---|---|
| 1 | 11 | 51 |
| 2 | 28 | 68 |
| 4 | 48 | 88 |
| 10 | 61 | 100 |

when tested by a specified test method which comprises using Ph.Eur. basket dissolution apparatus at 37° C., 100 rpm in 900 ml of simulated intestinal fluid at pH 6.8 without enzyme.

41. A pharmaceutical composition according to claim 40, wherein the release rates of oxycodone meet the following lower and upper limits:

| Hour | % Released Lower Limit | % Released Upper Limit |
|---|---|---|
| 1 | 16 | 46 |
| 2 | 33 | 63 |
| 4 | 53 | 83 |
| 10 | 66 | 96 |

when tested by the specified test at pH 6.8.

42. A pharmaceutical composition according to claim 41, wherein the release rates of oxycodone meet the following lower and upper limits:

| Hour | % Released Lower Limit | % Released Upper Limit |
|---|---|---|
| 1 | 19 | 43 |
| 2 | 36 | 60 |
| 4 | 56 | 80 |
| 10 | 69 | 93 |

when tested by the specified test at pH 6.8.

43. A pharmaceutical composition according to claim 26, which is intended for administration at intervals of about 24 hours.

44. A pharmaceutical composition according to claim 43, wherein the unit dose form has an oxycodone dissolution rate in vitro, when measured by the USP Basket Method at 100 rpm in 900 ml aqueous buffer at a pH between 1.6 and 7.2 at 37° C. of from 0% to about 40% at 1 hour, from about 8% to about 70% at 4 hours, from about 20% to about 80% at 8 hours, from about 30% to about 95% at 12 hours, from about 35% to about 95% at 18 hours, and greater than about 50% at 24 hours.

45. A pharmaceutical composition according to claim 44, wherein the peak plasma level of oxycodone obtained in vivo is reached at about 2 hours to about 17 hours after administration, at steady state.

46. A method of providing pain relief which comprises administration of an effective amount of a pharmaceutical composition as defined in claim 26.

47. A method of providing analgesia which comprises administration of an effective amount of a pharmaceutical composition as defined in claim 26.

48. A process for preparing multiparticulates which comprises preparing a blend which contains (a) oxycodone, (b) water-insoluble ammonium methacrylate copolymer, (c) plasticiser, (d) lubricant and (e) water permeability modifier; and extruding the blend.

49. A pharmaceutical composition in unit dose form comprising multiparticulates according to claim 10, and multiparticulates of oxycodone antagonist.
